(19) United States
(12) Patent Application Publication
(10) Pub. No.: US 2010/0042227 A1
(43) Pub. Date: Feb. 18, 2010

(54) Prosthesis Comprising a Shank for Accommodating an Amputation Stump

(76) Inventor: Arno Schmidt, Bovenden (DE)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/443,057

(22) PCT Filed: Sep. 11, 2007
(86) PCT No.: PCT/DE07/01629

§ 371 Date: Mar. 26, 2009

(30) Foreign Application Priority Data
Sep. 26, 2006 (DE) 10 2006 046 928.3

(51) Int. Cl. A61F 2/80; A61F 2/78

(52) U.S. Cl. 623/36; 623/33

(57) ABSTRACT

The invention relates to a prosthesis comprising at least one artificial limb (6) of an extremity and a shank (2), connected to the artificial limb (6), comprising a proximal opening for accommodating an amputation stump. The aim of the invention is to reduce the weight of a prosthesis of the above kind and increase its wearing comfort. For this purpose, the shank (2) consists of a stable outer shank (3) constituting the connection to the artificial limb (6) and a flexible Inner shank (4). The outer shank (3) has a distal endpiece (6) and shell segments (7, 8) extending therefrom towards the proximal pats, forming a space (9) between them.

The invention relates to a prosthesis with at least one artificial limb of an extremity and a socket, connected to the artificial limb, having a proximal opening for accommodating an amputation stump.

Numerous embodiments of such prostheses which are suitable for patients with an amputated arm or leg have been known for a long time. The connection to the amputation stump is critical for the functioning of a prosthesis. The prosthesis connected to the amputation stump forms artificial limbs, for example a leg with an artificial foot, an artificial lower leg and a knee joint if the amputation was carried out on the thigh. By contrast, if the step of amputation was carried out on the lower leg, the artificial limb of the prosthesis is essentially an artificial foot and possibly a short artificial lower-leg part. Modifications are possible by virtue of the fact that it is also possible to amputate within a joint. This correspondingly applies to the conditions in the case of an arm prosthesis. The prostheses mentioned initially are suitable for permanent provision of the patient, that is to say they are designed for long-term use on an amputation stump which has healed after the amputation.

The prosthesis is attached to the amputation stump using a known technique by means of a stable socket which surrounds the amputation stump on all sides and which is designed in the shape of a regular funnel. It is used to transfer the force from the amputation stump to the at least one artificial limb of the prosthesis. This is not without difficulties because the amputation stump can have a significant proportion of soft tissue. The sockets, which used to be made from wood, have, for some time now, been made from fiber-reinforced plastics which have the required stability and can be produced in simplified production processes. Since such a socket must be stable, wearing often leads to problems on the amputation stump as a result of the hard socket. Although the provision of a pulled-over liner, which effects a certain cushioning effect, is known for the protection of the amputation stump, sensitive pressure points at the upper socket edge frequently occur despite the liner because said liners are connected to the socket at the upper edge of the socket by folding or the like. The weight of the arrangement is a further problem.

The present invention is based on the object of developing a prosthesis of the type mentioned initially in such a fashion that it is more comfortable to wear and can be designed with a reduced weight.

According to the invention, this object is achieved in the case of a prosthesis of the type mentioned initially by virtue of the fact that the socket comprises a stable outer socket providing the connection to the artificial limb and a flexible inner socket and by virtue of the fact that the outer socket has a distal end piece and shell segments which extend proximally therefrom and form an intermediate space between them.

Hence, the prosthesis according to the invention has a socket which is of two-part design, with it preferably being possible for the inner socket and the outer socket to be interlocked, in particular as a result of a radial projection on the inner socket which interacts with a corresponding cut-out of the outer socket in order to in this manner prevent a relative extraction motion and relative rotation of the inner socket with respect to the outer socket. Here, the function of the inner socket is to closely surround the amputation stump stably and with an even force, while the outer socket no longer needs to perform this function and only ensures the stability of the connection to the artificial limb of the prosthesis by preferably only partially surrounding the amputation stump.

The outer socket has a distal end piece with a connection for the at least one artificial limb and shell segments which extend proximally from said end piece, the shell segments forming an intermediate space between them. For reasons of stability, the distal end piece is preferably designed as a closed cup. The outer socket clasps the amputation stump using the stability of its material and provides the required stable hold for the prosthesis wearer.

The shell segments can preferably be two shell segments which lie opposite one another, although it goes without saying that three or four shell segments are also possible.

The shell segments are preferably designed for—partially—clasping the amputation stump medially and laterally. However, it is also possible to form two shell segments lying opposite one another which partially clasp the amputation stump anteriorly and posteriorly.

In a particularly preferred embodiment of the invention, at least one shell segment is connected to the distal end piece such that it can swivel against an elastic resistance from an initial position. To this end, a shape can be used which forms a type of film hinge between the shell segment which can swivel and the distal end piece so that the arrangement of the shell segment which can swivel against the elastic restoring force is attained only by the shape, i.e. without additional elements.

In the preferred embodiment of the outer socket with a medial and a lateral shell segment, the medial shell segment is fixedly connected to the distal end piece and the lateral shell segment is connected to the distal end piece such that it can swivel against the elastic resistance.

By forming at least one shell segment which can swivel, it is possible for the stable outer socket, which provides for the transmission of the force, to also automatically adapt the volume in response to changes of the amputation stump, while keeping its stable and the feeling of a secure force transmission between amputation stump and prosthesis. It is known that, when wearing a prosthesis, a loss of bodily fluids in the amputation stump easily leads to a reduction in volume if the prosthesis is worn for a relatively long time during the day. This reduction in volume can be compensated for by the shell segment which can swivel, just like a possible swelling of the amputation stump which occurs in particular in recent amputees. The socket arrangement according to the invention thus has a variable volume in the proximal region, on the one hand as a result of the flexible inner socket and on the other hand as a result of the outer socket being provided with at least one shell segment which can swivel. Suitable sizing of the swivel resistance results in coupling between the amputation stump and the artificial limb of the prosthesis remaining approximately constant.

A frame-like structure already results from the intermediate space between the shell segments. However, the shell segments themselves can also be designed in the form of a frame with at least one cut-out. Preferably, a locking lug of the inner socket engages in an interlocking fashion in a cut-out of a shell segment.

So as to adapt the volume by swiveling the shell segment which can swivel with respect to the fixed shell segment, provision can be made for a tension element, which limits this relative swivel motion, to bridge the intermediate space between the shell segments. The tension element preferably comprises two band sections which can be connected to one another by a fastener and are preferably inelastic so that the effective length of the tension element can be adjusted. For the purposes of a simple implementation of such a fastener, the band sections can be designed as hook and loop elements. Hence, the tension element does not prevent the relative movability of the shell segments for the purposes of adapting the volume; rather it is only used as a limit against swiveling out too far.

The inner socket preferably comprises a flexible material which is inelastic, at least in the region supporting the load of the prosthesis wearer, and which does not noticeably elongate as a result of the weight load of the patient and also does not cause transverse strain in the case of occurring rotational motion; as a result of this, the outer socket also moves in an unretarded fashion in the case of rotational motion of the amputation stump due to the locking between inner socket and outer socket so that a torque resulting from rotational motion is transferred directly.

The inner socket can be designed to be closed at its distal end lying opposite the proximal opening. It preferably has an elastically designed end edge on the proximal opening which delimits this opening; as a result of this a comfortable end of the inner socket with respect to the amputation stump is ensured.

The inner socket can comprise a material that can be rolled up onto the amputation stump so that the inner socket can be fastened to the amputation stump when the inner socket is inside-out and can then be rolled over the amputation stump in the correct position.

Alternatively, it is possible for the inner socket to be provided with a closable, axially aligned opening slit which can, for example, be closed by a zipper or a hook and loop fastener. In this case, the inner socket is fastened to the amputation stump when the inner socket is open and, after attachment to the amputation stump, said socket is closed by means of the zipper, the hook and loop fastener or a similar closing element.

The lock between the inner socket and outer socket is preferably effected by means of at least one locking lug which is integrally formed onto the outer side of the inner socket.

In a preferred embodiment of the invention, the locking lug can have a proximally increasing ramp bevel which merges into a locking edge which falls away and hence has a sawtooth-like shape. The inner socket is then locked to the outer socket by patients who, having affixed the inner socket, load the outer socket using their weight, as a result of which the inner socket moves downward relative to the outer socket and so the locking lug with a sawtooth-like shape is pushed behind a corresponding locking edge of the outer socket and hence an extraction motion of the inner socket with respect to the outer socket is prevented.

The inner socket is expediently composed of a fabric-reinforced plastic, with it being possible to use all suitable plastics, in particular silicone, polyurethane, etc. The inner socket can also be formed by two layers, and can, for example, have fabric on the inner side or on the outer side. By way of example, the fabric material can be formed from polyester threads, Kevlar threads, etc. with arbitrary textures. Knitted fabrics, which are usual in liners to allow the latter's elasticity, are not suitable.

In order to implement elasticity in the edge end of the proximal opening of the inner socket, said end edge is designed without fabric reinforcement. Furthermore, it is advantageous if the end edge is designed to have a material thickness which decreases toward the opening so that the end of the inner socket on the amputation stump is even more comfortable. Incidentally, the inner socket preferably significantly protrudes above the upper end of the outer socket. The inner socket according to the invention is connected to the outer socket, in particular by means of the locking lugs, at a significant distance from the upper end of the outer socket and preferably in the central region of the outer socket in the axial direction. The outer socket is preferably formed by a frame construction with a plurality of cut-outs. This is possible because the outer socket only has a force-transmitting function and it is no longer necessary for said outer socket to surround the amputation stump from all sides. This makes it possible to save significant amounts of material in and significantly reduce the weight of the socket structure.

The outer socket is preferably composed of stable, fiber-reinforced plastic which has the required stiffness.

The invention is intended to be explained in more detail below on the basis of exemplary embodiments illustrated in the drawing, in which

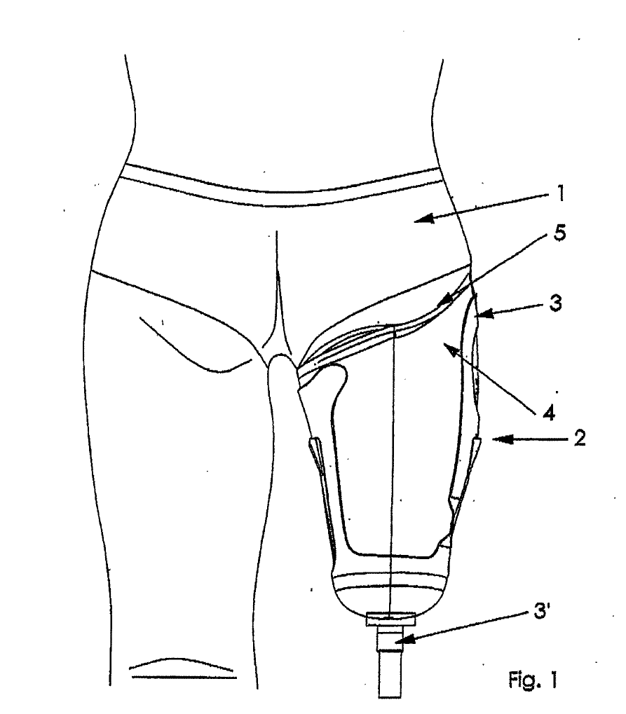

FIG. 1 shows a back view of a patient with an affixed prosthesis according to an embodiment of the invention;

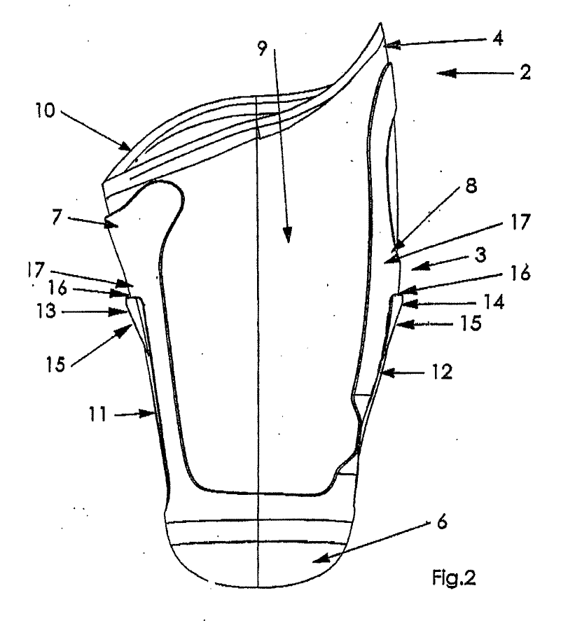

FIG. 2 shows a corresponding view of the socket arrangement of the prosthesis in accordance with FIG. 1;

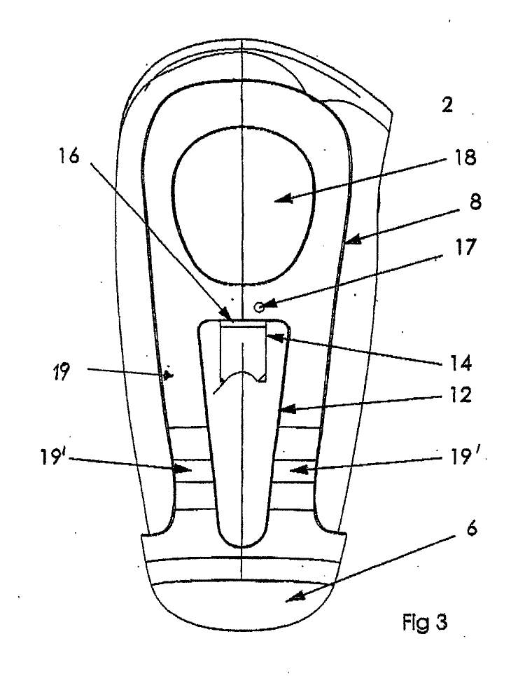

FIG. 3 shows a lateral view of the socket arrangement;

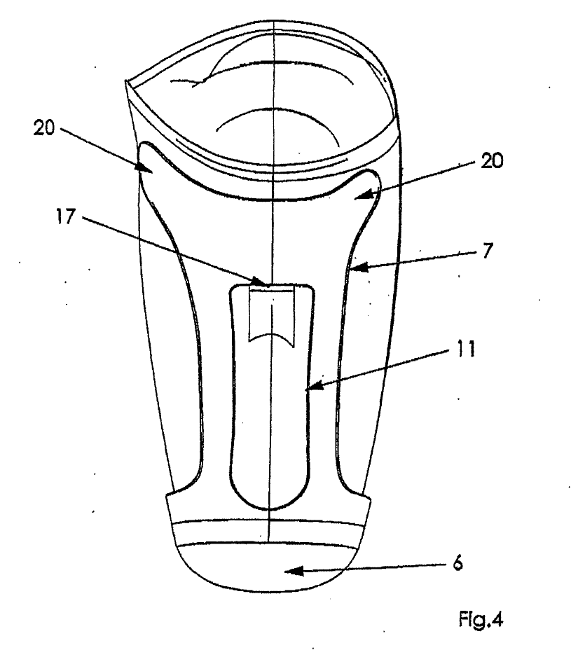

FIG. 4 shows a medial view of the socket arrangement;

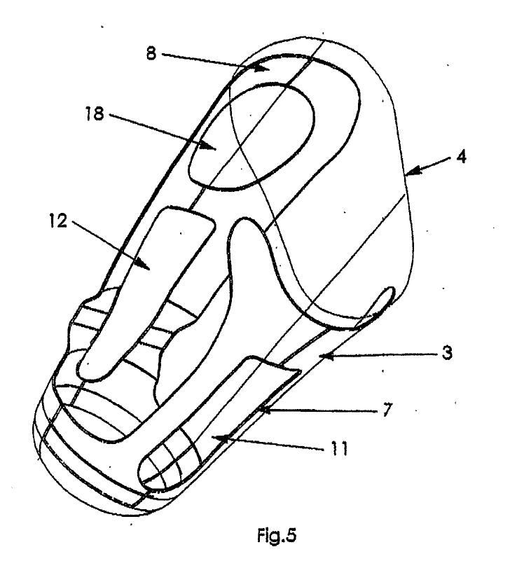

FIG. 5 shows a perspective view of the socket arrangement;

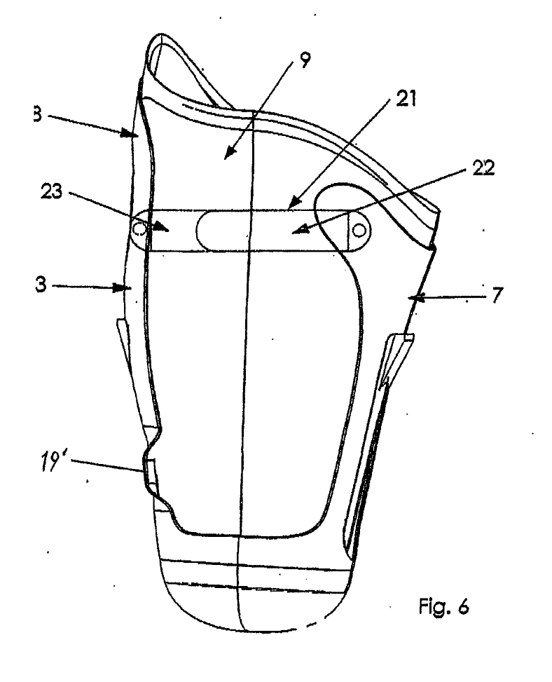

FIG. 6 shows a back view of a modified socket arrangement;

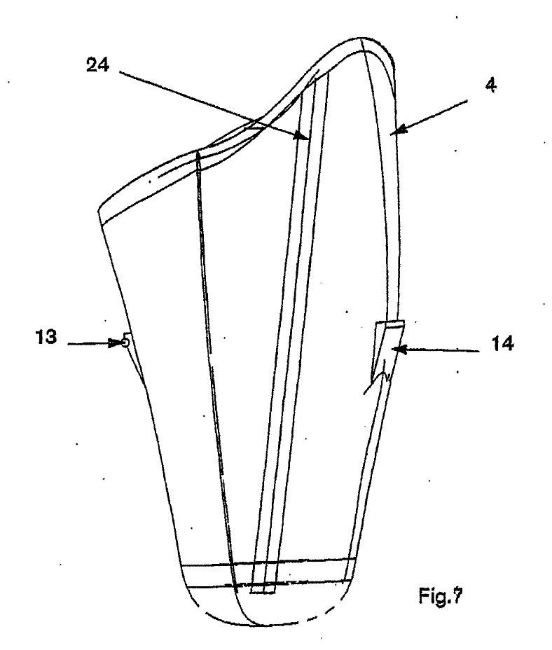

FIG. 7 shows a view of a modified inner socket with an axial opening slit; and

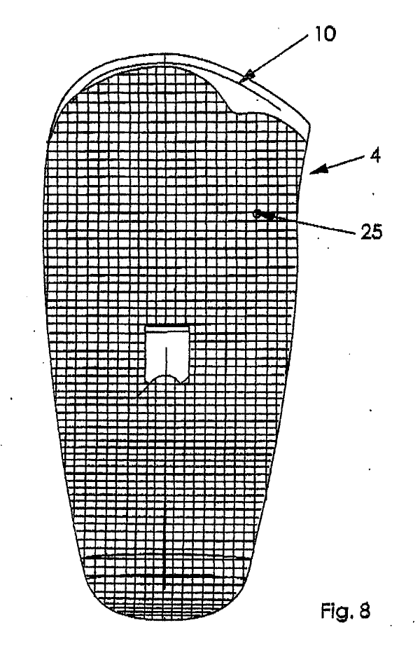

FIG. 8 shows an illustration to clarify the fabric reinforcement of the inner socket.

FIG. 1 shows the back view of a patient 1 with an amputation stump (not illustrated) on the thigh. The amputation stump is inserted into a socket arrangement 2 comprising an outer socket 3 and an inner socket 4.

The socket arrangement 2 surrounds the amputation stump, which is inserted through a proximal opening 5 of the inner socket 4, in a funnel-like fashion.

At the distal end, a module pipe 3′ adjoins the outer socket 3 as part of an artificial leg.

FIG. 1, and also FIG. 2, show that the outer socket 3 has a distal end piece 6, designed as a closed cup, from which two shell segments 7, 8 extend upward and form an intermediate space 9 between them.

The outer socket 3 accommodates the inner socket 4, with the latter extending proximally above the upper end of the outer socket 3 and forming an end edge 10 there.

Since the views in FIGS. 1 and 2 are dorsal views (back views), the shell segments 7, 8 lying opposite one another are a (shorter) medial shell segment 7 and a (longer) lateral shell segment 8. The two shell segments 7, 8 have slit-like cut-outs 11, 12, in which the locking lugs 13, 14, arranged on the outer side of the inner socket 4, engage in an interlocking manner. The locking lugs 13, 14 each form a ramp bevel 15 which increases from the bottom to the top (from distal to proximal) and which, at the proximal end, merges into a locking edge 16 which engages in an interlocking manner behind an upper limit 17 of the respective shell segment 7, 8. In the illustrated cross section, the locking lugs 13, 14 thus form a sawtooth shape.

The interlocking mechanism is made even clearer in the side view of the lateral shell segment 8 in FIG. 3. The locking lug engages in the slit-shaped cut-out 12 and butts against the upper end 17 of the lateral shell segment with its locking edge 16. Above this, the lateral shell segment 8 also has a cut-out 18 in the shape of an ellipse which is used to save weight and material. Hence, the lateral shell segment 8 is in the form of a frame and has a central cross web which constitutes the upper limit 17 of the lower slit-shaped cut-out 12.

The drawing shows that the lateral shell segment 8 is connected to the end piece 6 via an introduced material weakening 19′ at the lower end of the webs 19 which merge into the end piece and delimit the slit-shaped cut-out 12 such that the lateral shell segment 8 can be swiveled to the outside through a certain angle against the restoring elasticity of the material.

By contrast, the medial shell segment 7 which can be seen in FIG. 4 is fixedly connected to the end piece 6. Since the medial shell segment 7 is shorter than the lateral shell segment 8, it only has one slit-shaped cut-out 11. The piece of material forming the upper limit 17 of the slit-shaped cut-out 11 is provided with shell-shaped, bent, finger-like projections 20 which engage around part of the amputation stump of the patient 1 on the medial side. The narrower region of the medial shell segment 7 provided with the slit-shaped cut-out also has a corresponding curvature.

This design of the outer socket 3 with the only schematically sketched inner socket 4 is once again made clearer in the perspective view of FIG. 5.

FIG. 6 shows a modification of the outer socket 3, which modification only consists of the swivel motion of the lateral shell segment 8 with respect to the fixed medial shell segment 7 being limited by a tension element 21 which bridges the intermediate space 9. The tension element 21 comprises two band sections 22, 23, which are attached to the lateral shell segment 8 and the medial shell segment 7, respectively, and the free ends of which can be connected to one another, the wall sections to this end being designed as parts of a hook and loop fastener. This makes it possible to set the maximum swivel angle of the lateral shell segment 8 relative to the fixed medial shell segment 7.

FIG. 7 shows a view of the inner socket 4 which in this case is provided with an opening slit 24 aligned in the axial direction. The opening slit can be formed by a zipper as fastening element or can be closed by a hook and loop fastener. The opening of the opening slit 24 makes it possible for the patient 1 to place the inner socket 4 against the amputation stump in a comfortable manner. Subsequently, the amputation stump with the applied inner socket 4 can be inserted into the outer socket 3 until it is locked by the locking lugs 13, 14.

FIG. 8 clarifies that the inner socket 4 preferably comprises a material formed with a reinforcement fabric 25. The reinforcement fabric can be found in the flexible plastic material, or else it can be applied to the outer side or the inner side of the plastic material.

It can be seen that the reinforcement fabric ends in front of the end edge 10 so that the end edge 10 can be designed to be elastic, while the inner socket 4 is flexible, but not elastic, in its remaining region which takes the load of the patient, as a result of the material reinforced by the reinforcement fabric 25.

The elasticity of the end edge 10 can be further increased by the end edge 10 tapering toward its upper end with a continuously decreasing material thickness; this results in comfortable and complete clinging of the end edge 10 to the skin of the amputation stump.

1. A prosthesis with at least one artificial limb (6) of an extremity and a socket (2), connected to the artificial limb (6), having a proximal opening (5) for accommodating an amputation stump, characterized in that the socket (2) comprises a stable outer socket (3) providing the connection to the artificial limb (6) and a flexible inner socket (4) and in that the outer socket (3) has a distal end piece (6) and shell segments (7, 8) which extend proximally therefrom and form an intermediate space (9) between them.

2. The prosthesis as claimed in claim 1, characterized in that the distal end piece (6) is designed as a closed cup.

3. The prosthesis as claimed in claim 1, characterized in that at least one shell segment (7, 8) is connected to the distal end piece (6) such that it can swivel against an elastic resistance from an initial position.

4. The prosthesis as claimed in claim 3, characterized in that the shell segment (8) which can swivel is connected to the distal end piece (6) by means of a film hinge.

5. The prosthesis as claimed in claim 1, characterized by two shell segments (7, 8) which lie opposite one another.

6. The prosthesis as claimed in claim 5, characterized in that the shell segments (7, 8) are designed for clasping the amputation stump medially and laterally.

7. The prosthesis as claimed in claim 6, characterized in that the medial shell segment (7) is fixedly connected to the distal end piece (6) and the lateral shell segment (8) is connected to the distal end piece (6) such that it can swivel against an elastic resistance.

8. The prosthesis as claimed in claim 1, characterized in that the shell segments (7, 8) themselves are designed in the form of a frame with at least one cut-out (11, 12, 18).

9. The prosthesis as claimed in claim 1, characterized in that the inner socket (4) comprises a material which is inelastic, at least in the region supporting the load.

10. The prosthesis as claimed in claim 1, characterized in that the inner socket (4) is closed at a distal end.

11. The prosthesis as claimed in claim 1, characterized in that the inner socket (4) has an elastically designed end edge (10) which delimits the proximal opening (5).

12. The prosthesis as claimed in claim 1, characterized in that the inner socket (4) comprises a material that can be rolled up.

13. The prosthesis as claimed in claim 1, characterized in that the inner socket (4) has a closable, axially aligned opening slit (24).

14. The prosthesis as claimed in claim 13, characterized in that the opening slit (24) is designed such that it can be closed by a zipper.

15. The prosthesis as claimed in claim 13, characterized in that the opening slit (24) is designed such that it can be closed by a hook and loop fastener.

16. The prosthesis as claimed in claim 1, characterized in that at least one locking lug (13, 14) is formed onto one outer side of the inner socket (4) as a projection for interlocking with the outer socket (3).

17. The prosthesis as claimed in claim 16, characterized in that the locking lug (13, 14) has a proximally increasing ramp bevel (15) which merges into a declining locking edge (16).

18. The prosthesis as claimed in claim 1, characterized in that the inner socket (4) is composed of a fabric-reinforced plastic.

19. The prosthesis as claimed in claim 11, characterized in that the end edge (10) is designed without fabric reinforcement (25).

20. The prosthesis as claimed in claim 19, characterized in that the end edge (10) is designed to have a material thickness which decreases toward the opening (5).

21. The prosthesis as claimed in claim 1, characterized in that the outer socket (3) is composed of a stable, fiber-reinforced plastic.

22. The prosthesis as claimed in claim 1, characterized in that the shell segments (7, 8) themselves are designed in the form of a frame with at least one cut-out (11, 12, 18).

23. The prosthesis as claimed in claim 16, characterized in that a locking lug of the inner socket (4) engages in an interlocking fashion in a cut-out (11, 12) of a shell segment (7, 8).

24. The prosthesis as claimed in claim 3, characterized in that a tension element (21), which limits the relative swivel motion of the shell segment (8) which can swivel compared to a fixed shell segment (7), bridges the intermediate space (9) between these shell segments (7, 8).

25. The prosthesis as claimed in claim 24, characterized in that the tension element (21) comprises band sections (22, 23) which can connected to one another by a fastener.

26. The prosthesis as claimed in claim 25, characterized in that the band sections (22, 23) are designed as hook and loop fastener elements.
